(19) United States
(12) Patent Application Publication
(10) Pub. No.: US 2022/0088585 A1
(43) Pub. Date: Mar. 24, 2022

(54) TEST STRIP, AND MICROBIAL SENSOR DEVICE AND SENSING METHOD

(71) Applicant: KOREA INSTITUTE OF SCIENCE AND TECHNOLOGY, Seoul (KR)

(72) Inventors: Joonseok LEE, Seoul (KR); Inae LEE, Seoul (KR)

(21) Appl. No.: 17/117,131

(22) Filed: Dec. 10, 2020

(30) Foreign Application Priority Data
Sep. 18, 2020 (KR) 10-2020-0120625

(51) Int. Cl. B01L 3/00; G01N 1/22

(52) U.S. Cl. CPC B01L 3/5023; B82Y 35/00; G01N 1/2273

(57) ABSTRACT

A test strip may include a porous sampling pad through which the air passes to collect a specimen, a conjugation pad which is positioned on a supporter in contact with the sampling pad, and in which a plurality of capture agent-nanoparticle composites specifically binding to a target material are dispensed, a membrane which is positioned on the supporter in contact with the conjugation pad, and includes a test line to which the capture agent-nanoparticle composites to which the target material binds are conjugated when the specimen moves and a control line to which the capture agent-nanoparticle composites are conjugated, and an absorption pad configured to absorb the moving specimen on the supporter.

### CROSS-REFERENCE TO RELATED APPLICATION

This application claims priority to and the benefit of Korean Patent Application No. 10-2020-0120625 filed in the Korean Intellectual Property Office on Sep. 18, 2020, the entire contents of which are incorporated herein by reference.

### BACKGROUND

### (a) Technical Field

The present disclosure relates to a test strip, a microbial sensor device including the same, and a method of sensing a microorganism.

### (b) Description of the Related Art

Severe acute respiratory syndrome (SARS, 2003), influenza A H1N1 virus 2009, Middle East respiratory syndrome coronavirus (MERS-CoV, 2005), and the like are known to be contagious diseases that are associated with the diffusion of pathogens in the air, cause tens of thousands of cases of infections, and kill hundreds of people. To prevent the infections and damage caused by these air-borne pathogens, it is important to detect them rapidly and effectively to prevent them from being diffused rapidly.

To effectively detect pathogens in the air, it is necessary to efficiently collect and analyze an atmospheric sample.

A conventional atmospheric sample is collected using a gravity settling method, an impactor method using a cascade impactor, an electrostatic method, a cyclone, and the like. In this case, heavy and large equipment is required, or it takes a long time to collect an atmospheric sample.

Also, such sampling methods require an additional processing process to analyze a certain hazardous material. In the case of microorganism, the analysis of the hazardous material in the atmospheric sample is performed by a culture method, mass spectrometry, a reverse-transcriptase polymerase chain reaction (RT-PCR), and the like. In this case, the culture method has drawbacks in that it requires a long time, and has limitations on quantification, and the methods using analysis equipment is sensitive and accurate, but has drawbacks in that it requires lots of time and cost due to the complicated pretreatment and expensive equipment. Therefore, these methods are not suitable for monitoring pathogens floating in a site.

The above information disclosed in this Background section is only for enhancement of understanding of the background of the invention, and therefore it may contain information that does not form the prior art that is already known in this country to a person of ordinary skill in the art.

### SUMMARY

An object of the present invention is to provide a measuring device for collecting and analyzing pathogens in the air to monitor the presence/absence and a concentration of the pathogens in a surrounding environment, and a measuring method thereof.

Another object of the present invention is to provide a device for collecting and detecting air-borne pathogens, which it is easy to carry and handle by means of a down-sized and integrated diagnosis platform according to the present invention, and a method of detecting the air-borne pathogens.

The combined sampling/diagnosis kit is combined with a small air sampling machine and analysis equipment so that it includes a device and a measuring method for performing in situ collection and detection at the same time.

Also, a platform for collecting and detecting a number of hazardous materials using a plurality of different combined sampling/diagnosis kits is suggested.

A test strip according to one aspect of the present invention includes a porous sampling pad through which the air passes to collect a specimen, a conjugation pad which is positioned on a supporter in contact with the sampling pad, and in which a plurality of capture agent-nanoparticle composites specifically binding to a target material are dispensed, a membrane which is positioned on the supporter in contact with the conjugation pad, and includes a test line to which the capture agent-nanoparticle composites to which the target material binds are conjugated when the specimen moves and a control line to which the capture agent-nanoparticle composites are conjugated, and an absorption pad configured to absorb the moving specimen on the supporter.

The sampling pad, the conjugation pad, the membrane, and the absorption pad may be positioned to be connected in this order along a moving direction of the specimen.

A portion of the sampling pad may be positioned on a portion of the conjugation pad so that the sampling pad and the conjugation pad partially overlap each other in a direction vertical to the moving direction.

A portion of the membrane may be positioned between a portion of the conjugation pad and the supporter so that the membrane and the conjugation pad partially overlap each other in a direction vertical to the moving direction.

A portion of the membrane may be positioned between a portion of the absorption pad and the supporter so that the membrane and the absorption pad partially overlap each other in a direction vertical to the moving direction.

When an analysis solution is dispensed in the sampling pad so that the specimen moves towards the conjugation pad along with the analysis solution, the target material in the specimen may bind to any one of the plurality of capture agent-nanoparticle composites.

A plurality of capture antibodies may be fixed in the test line, and one of the plurality of capture antibodies may bind to the target material of the capture agent-nanoparticle composites to which the target material binds.

A plurality of control antibodies may be fixed in the control line, the control line may be positioned behind the test line in a moving direction of the specimen, and one of the plurality of control antibodies may bind to the capture agent of the capture agent-nanoparticle composites.

The capture agent-nanoparticle composites may absorb infrared rays with a first wavelength to emit infrared rays with a second wavelength, and the first wavelength may be longer than the second wavelength.

Each of the conjugation pad, the membrane, and the absorption pad may include a solid-phase capillary support, and the porosity of the sampling pad may be greater than the porosity of the solid-phase capillary support.

A surface of the sampling pad may be treated with a mixed solution of PVP, sucrose, BSA, and Tween 20.

A microbial sensor device according to another aspect of the present invention includes an air sampling device configured to collect air, a combined sampling/diagnosis kit positioned on an upper surface of the air sampling device to collect a specimen from the air sucked by the air sampling device, and an analysis device configured to irradiate the kit with infrared rays with a first wavelength to receive the infrared rays with a first wavelength from the combined sampling/diagnosis kit, thereby detecting a target material from the specimen. The combined sampling/diagnosis kit includes a porous sampling pad configured to collect the specimen from the air passing through the corresponding one air suction port, a conjugation pad which is positioned in contact with the sampling pad, and in which a plurality of capture agent-nanoparticle composites specifically binding to the target material are dispensed, a membrane which is positioned in contact with the conjugation pad, and includes a test line to which the capture agent-nanoparticle composites to which the target material binds are conjugated when the specimen moves, and a control line to which the capture agent-nanoparticle composites are conjugated, and an absorption pad configured to absorb the moving specimen.

The air sampling device includes an air suction port device configured to provide an air path through which air flowing in through an air suction port of the combined sampling/diagnosis kit flows, a hollow air suction device configured to collect the air passing through the air path, and an air suction fan device configured to produce an air suction force. The sampling pad may be positioned in the air path.

The air suction port device may include an air outlet formed in a position corresponding to the air suction port of the combined sampling/diagnosis kit, and an air suction through hole formed to extend from the air outlet, and the sampling pad may be positioned on the air suction through hole.

The hollow air suction device may include a first circular aperture formed on an upper surface thereof, a second circular aperture formed on a lower surface thereof, and a common suction space formed between the first circular aperture and the second circular aperture.

The common suction space includes a circular cylinder space having a predetermined depth from the first circular aperture, and a space having a trapezoid circular cylinder shape between a third circular aperture positioned in a lower portion of the circular cylinder space and the second circular aperture.

The air suction fan device includes a fan configured to produce the air suction force while rotating, and an outlet configured to discharge the air sucked by rotation of the fan.

The combined sampling/diagnosis kit may further include a cartridge configured to accommodate a test strip in order to couple the test strip, which includes the supporter, the sampling pad, the conjugation pad, the membrane, and the absorption pad, to the air sampling device.

A method of detecting a microorganism using the kit coupled to the air sampling device according to still another aspect of the present invention includes collecting a specimen from air sucked by the air sampling device using a porous sampling pad through which the air passes, dispensing an analysis solution in the sampling pad to move the specimen thereto, conjugating a target material included in the specimen to one of a plurality of capture agent-nanoparticle composites in a conjugation pad, conjugating a capture antibody fixed in a membrane to the target material of the capture agent-nanoparticle composites to which the target material binds as the specimen moves, and absorbing the moving specimen using an absorption pad.

The method of detecting a microorganism may further include conjugating at least one of the plurality of capture agent-nanoparticle composites to a control antibody fixed in the membrane.

The present invention can monitor the presence/absence and a concentration of the hazardous microorganisms is situ in a surrounding environment in real time by combining a small air sampling machine, a combined sampling/diagnosis kit, and portable analysis equipment to collect and analyze microorganisms in the air in real time.

Also, the present invention can provide a device for collecting and detecting an air-borne microorganism, which is easy to carry and handle with down-sizing of the equipment, and a method of detecting the air-borne microorganism.

Further, the present invention can be applied to a measuring device and a method of collecting and analyzing contaminant microorganisms in the air, water, and foods, and other contaminants hazardous to the human body in real time to detect the presence/absence and a concentration of the contaminant microorganisms and the other contaminants, thereby making it possible to take rapid action for and prevent the air contaminants.

### BRIEF DESCRIPTION OF THE DRAWINGS

FIG. 1 is a perspective view showing a microbial sensor device according to an exemplary embodiment.

FIG. 2 is a diagram schematically showing a microbial sensor device according to an exemplary embodiment.

FIG. 3 is an exploded diagram of an air sampling device according to an exemplary embodiment.

FIG. 4 is a diagram schematically showing a plurality of air suction through holes and an inner space of the air sampling device.

FIG. 5 is a graph showing an average flow rate of air flowing through each of the plurality of air suction through holes.

FIG. 6 is a graph showing a concentration of nano-micro particles collected and concentrated in detection pads of four combined sampling/diagnosis kits.

FIG. 7 is a graph showing a concentration of viruses collected and concentrated in the detection pads of four combined sampling/diagnosis kits.

FIG. 8 is a diagram showing various arrays of a plurality of combined sampling/diagnosis kits in a common suction space.

FIG. 9 is a diagram showing a combined sampling/diagnosis kit according to an exemplary embodiment.

FIG. 10 is an exploded diagram showing a structure of the combined sampling/diagnosis kit according to an exemplary embodiment.

FIG. 11 is a perspective view showing a test strip according to an exemplary embodiment.

FIGS. 12 to 14 are perspective cross-sectional views of a combined sampling/diagnosis kit for explaining an operation of the combined sampling/diagnosis kit.

FIG. 15 is a graph showing the results of detection of MS2 viruses using the combined sampling/diagnosis kit coupled to the air sampling device.

FIG. 16 is a graph showing a recovery rate of MS2 viruses.

FIG. 17 is a graph showing a detection ratio of the MS2 viruses recovered from a sampling pad.

FIG. 18 is a diagram schematically showing a chamber for an experiment used to collect air and detect a target material.

FIG. 19 is a diagram showing the results of evaluating collection performance of the microbial sensor device to which the combined sampling/diagnosis kit is coupled.

FIG. 20 is a graph showing the results of detection of MS2 viruses depending on a sampling time and a flow rate in the microbial sensor device.

FIG. 21 is a graph showing the results of detection of avian influenza virus using the microbial sensor device.

FIG. 22 shows the results of collection and detection of avian influenza virus in the air using the microbial sensor device.

FIG. 23 shows the results showing diagnostic selectivity to the avian influenza virus in the microbial sensor device.

### DETAILED DESCRIPTION OF THE EMBODIMENTS

Because air-borne pathogens causing contagious diseases spread easily, a monitoring system capable of detecting and identifying the pathogens in situ may be helpful to prevent and control the diffusion of the pathogen at an early stage. That is, there is need for a diagnosis platform that is rapid and accurate, and easy to use when the diagnosis platform is coupled to an atmospheric sample collecting machine.

Paper-based lateral flow immunoassay (LFA) chromatography analysis is a platform for in situ diagnosis (hereinafter referred to as “LFA platform”) that may show simple, rapid, and sensitive diagnosis results of various viruses. However, the LFA platform is composed of a method of dispensing a sample in an aqueous solution on a sampling pad and analyzing the sample. That is, a large amount of a sample may be lost while collecting an atmospheric sample in the existing air sampling machine, recovering the sample in a solution phase, and then moving the recovered sample to the LFA platform. This may result in deteriorated accuracy and sensitivity of the diagnosis system.

As the LFA platform according to an exemplary embodiment, an integrated LFA platform capable of spatially combining collection and analysis of the sample in order to minimize the loss of the sample is provided.

That is, the integrated LFA platform according to an exemplary embodiment is an integrated sampling and detection platform for detecting pathogens floating in the air is situ in real time, and may include an air sampling device, a paper-based combined sampling/diagnosis kit, and a portable analysis device.

The integrated LFA platform according to an exemplary embodiment may be easy to carry due to the down-sizing of the air sampling device and the analysis device. Also, because the integrated LFA platform is combined as one platform, detection results may be rapidly obtained by sampling and analyzing a specimen in situ.

A plurality of sampling units are composed in the integrated LFA platform, and it is possible to collect and detect a plurality of different pathogens at the same time by combining the combined sampling/diagnosis kit, in which diagnostic capture agents specific to different pathogens are used, to the platform.

Registered Korean Patent No. 10-2049946 may be referenced to the known technology which is not described in the present disclosure.

Hereinafter, Examples disclosed in this specification will be described in detail with reference to the drawings, where like or similar constituent elements have like or similar reference numerals, and thus a repeated description thereof will be omitted.

The suffixes “module” and/or “unit” of the constituent element used in the following description are given in consideration of the easy drafting of this specification only, or are used interchangeably, and not intended to have the meanings or roles that are distinguished from each other in themselves. Also, in describing the Examples disclosed in this specification, a specific description of the related technology known in the art is judged to make the gist of the Examples disclosed in this specification unclear, a detailed description thereof is omitted. Also, the accompanying drawings are merely intended to easily understand the Examples disclosed in this specification, but the accompanying drawings are not intended to limit the technical scope of the Examples disclosed in this specification. Therefore, it should be understood that the present invention encompasses all changes, modifications, equivalents and substitutions which fall within the scope and spirit of the present invention.

The terms including the ordinal numbers such as first, second, and the like may be used to explain various constituent elements, but the terms are not intended to limit the constituent elements. The terms are used to differentiate one constituent element from another constituent element.

It is said that any constituent element is “connected to” or “in contact with” another constituent element, it should be understood that the any constituent element may be directly connected to or in direct contact with another constituent element, but may be connected to the another constituent element through another constituent element. On the other hand, it is said that any constituent element is “directly connected to” or “in direct contact with” another constituent element, it should be understood that there is no another constituent element between them.

In this application, the term “comprising”, “having” or the like is used to specify that a feature, a number, a step, an operation, a constituent element, a part, or a combination thereof disclosed in this specification is present, but does not exclude the possibility of presence or addition of one or more other features, numbers, steps, operations, constituent components, parts or combinations thereof in advance.

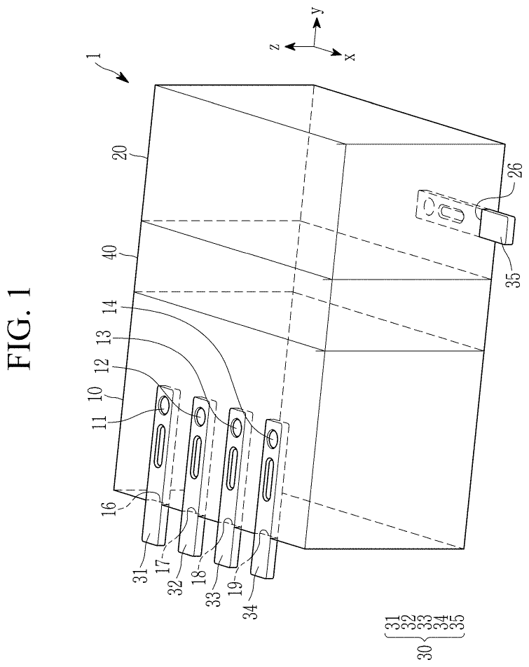

FIG. 1 is a perspective view showing a microbial sensor device according to an exemplary embodiment.

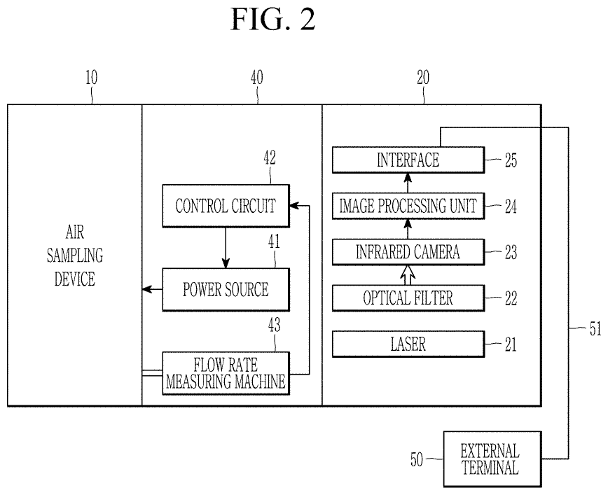

FIG. 2 is a diagram schematically showing a microbial sensor device according to an exemplary embodiment.

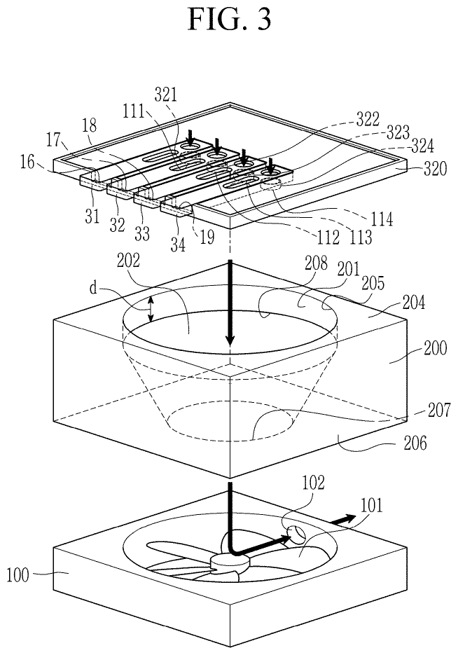

FIG. 3 is an exploded diagram of an air sampling device according to an exemplary embodiment.

As shown in FIGS. 1 to 3, a microbial sensor device 1 includes an air sampling device 10, an air sampling drive unit 40, an analysis device 20, and a plurality of combined sampling/diagnosis kits 30. The air sampling device 10 includes an air suction fan device 100, a hollow air suction device 200, and an air suction port device 300.

The air suction port device 300 provides four paths through which the air sucked through four combined sampling/diagnosis kits 31-34 may be sucked without air leakage. The number of the air flow paths is determined depending on the number of the combined sampling/diagnosis kits. The four combined sampling/diagnosis kits 31-34 are inserted and coupled to an upper portion of the air suction port device 300. The air suction port device 300 includes four air suction through holes 321-324 positioned to correspond to the air suction ports 11-14 of the four combined sampling/diagnosis kits 31-34.

Four outlets 111-114 are formed in the air suction port device 300 in positions corresponding to the air suction ports 11-14, and each of the four air suction through holes 321-324 is formed to extend from the corresponding outlet (one of 111-114) along the z axis.

The four combined sampling/diagnosis kits 31-34 are coupled to the air sampling device 10 through four coupling grooves 16-18, and each of sampling pads of the four combined sampling/diagnosis kits 31-34 is positioned on one corresponding hole of the four air suction through holes 321-324. The sampling pads of the four combined sampling/diagnosis kits 31-34 serve to collect a specimen from the air sucked through the four air suction through holes 311-314, and allow the air passing through the sampling pads to flow to the hollow air suction device 200 through the four air suction through holes 331-334 and the four air suction through holes 321-324.

In FIG. 1, the number of the suction ports, the air suction through holes, and the coupling grooves, and the number of the combined sampling/diagnosis kits capable of being coupled thereto is four, and may vary depending on their design, but the present invention is not limited thereto.

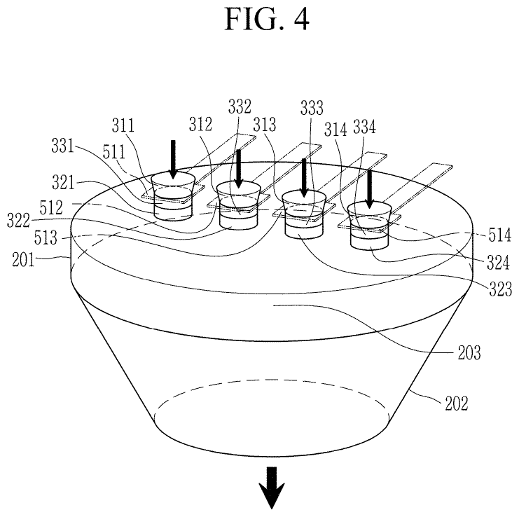

FIG. 4 is a diagram schematically showing a plurality of air suction through holes and an inner space of the air sampling device.

FIG. 4 shows configurations associated with the air flow in order to show that the flow of air sucked through a plurality of air suction through holes of the air sampling device is uniform. In FIG. 4, the four combined sampling/diagnosis kits 31-34 are coupled to the air suction port device 300 so that each of the sampling pads of the four combined sampling/diagnosis kits 31-34 is positioned on the corresponding air suction through hole (one of 321-324).

As shown in FIG. 4, when the air suction fan device 100 is operated, the air is sucked through the plurality of air suction through holes 311-314, and the sucked air is allowed to pass through sampling pads 510 of the four combined sampling/diagnosis kits 31-34, and collected in a common suction space 203 defined by an interior circumference 202 and an interior circumference 201 of the hollow air suction device 200 through the plurality of air suction through holes 321-324, and then sucked into the air suction fan device 100.

The hollow air suction device 200 includes a circular aperture 205 formed on an upper surface 204 thereof, a circular aperture 207 formed on a lower surface 206 thereof, and a common suction space 203 formed between the circular aperture 205 and the circular aperture 207. The common suction space 203 includes a circular cylinder space having a predetermined depth d from the circular aperture 205, and a space having a trapezoid circular cylinder shape between a circular aperture 208 positioned in a lower portion of the circular cylinder space and the circular aperture 207.

The air suction fan device 100 includes a fan 101 configured to produce an air suction force while rotating, and an outlet 102 configured to discharge the air sucked by rotation of the fan 101. As shown in FIG. 3, when the fan 101 rotates, the air passing through the four suction ports 11-14, the air suction through holes 311-314 formed in an upper lids of the four combined sampling/diagnosis kits 31-34, the sampling pads of the four combined sampling/diagnosis kits 31-34, the air suction through holes 331-334 formed in a lower lids of the four combined sampling/diagnosis kits 31-34, the four air suction through holes 321-324, and the common suction space 203 may be discharged out through the outlet 102.

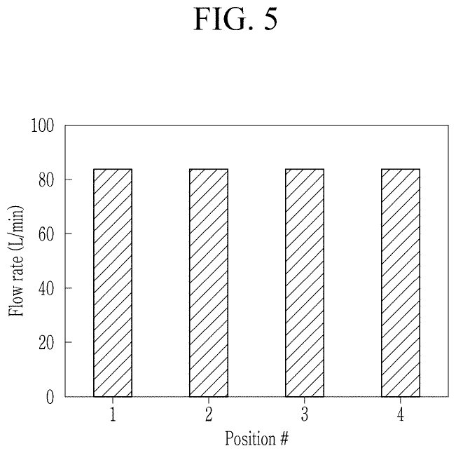

FIG. 5 is a graph showing an average flow rate of air flowing through each of the plurality of air suction through holes.

A plurality of air suction through holes 311-314, 331-334, 321-324 are numbered by positions, and the number indicating the corresponding number is a horizontal axis on the graph of FIG. 5. Number 1 is given to the engaged three air suction through holes 311, 331, and 321, Number 2 is given to the engaged three air suction through holes 312, 332, and 322, Number 3 is given to the engaged three air suction through holes 313, 333, and 323, and Number 4 is given to the engaged three air suction through holes 314, 334, and 324.

The vertical axis on the graph of FIG. 5 represents an air average flow rate, and its units is a liter per minute (L/min). As shown in FIG. 5, it can be seen that the average flow rate of the air flowing through the plurality of air suction through holes 311-314, 331-334, 321-324 in positions of Numbers 1 to 4 is uniform to be 80 L/min. Because the total amount of the air collected through the plurality of air suction through holes 311-314, 331-334, 321-324 is uniform, and an amount of the air passing through the plurality of combined sampling/diagnosis kits 31-34 is uniform, conditions for collecting a hazardous material using the plurality of combined sampling/diagnosis kits 31-34 may also be controlled uniformly.

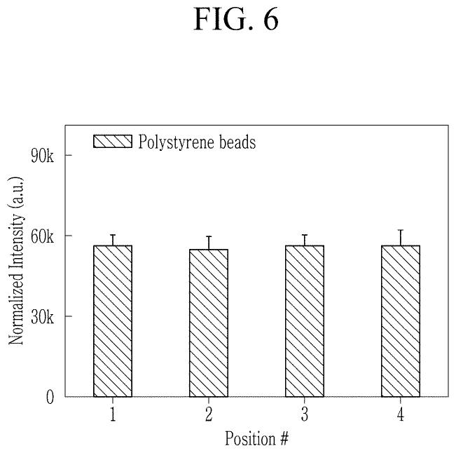

FIG. 6 is a graph showing a concentration of nano-micro particles collected and concentrated in detection pads of four combined sampling/diagnosis kits.

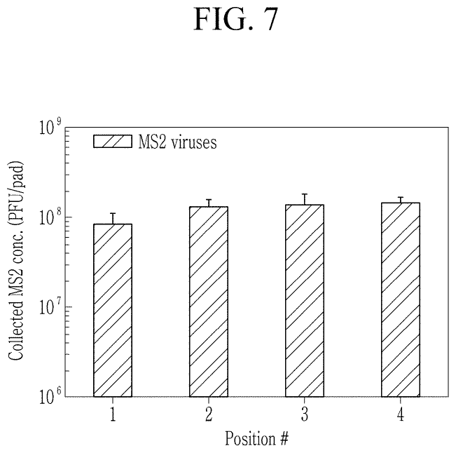

FIG. 7 is a graph showing a concentration of viruses collected and concentrated in the detection pads of four combined sampling/diagnosis kits.

In FIG. 6, concentrations of the nano-micro particles (polystyrene beads, 30 nm) collected and concentrated in the sampling pads 510 of the four combined sampling/diagnosis kits 31-34 are shown based on the positions (Numbers 1-4) of the combined sampling/diagnosis kits, respectively. The nano-micro particles may be fluorescent microparticles. As shown in FIG. 6, it can be seen that the concentrations of the nano-micro particles of Numbers 1-4 are uniform. The concentrations are represented by the normalized intensities, which are indicated in arbitrary units (a.u.).

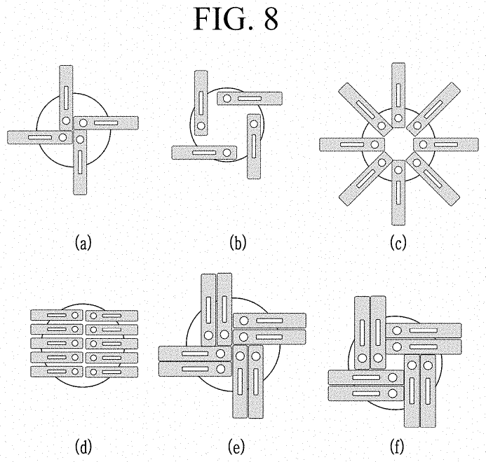

FIG. 7 shows the concentrations of the MS2 viruses collected and concentrated by the sampling pads 510 of the four combined sampling/diagnosis kits 31-34 based on the positions (Numbers 1-4) of the combined sampling/diagnosis kits, respectively. As shown in FIG. 8, it can be seen that the concentrations of the MS2 viruses spanning from Numbers 1 to 4 is roughly uniform. The concentrations are indicated in plaque forming unit (PFU) units (PFU/pad) in the sampling pads of the combined sampling/diagnosis kits.

As such, as can be seen in FIGS. 5 to 7, it can be seen that a uniform amount of the specimen is collected and concentrated through the four combined sampling/diagnosis kits 31-34, thereby making it possible to collect and detect a number of target materials in the specimen at the same time when the target materials are inserted into the four different combined sampling/diagnosis kits.

In an exemplary embodiment as shown in FIGS. 1 to 4, suction ports 111-114 are shown to be lined up in the middle of the common suction space, but the present invention is not limited thereto. The average flow rate of the air sucked through each of the plurality of air suction through holes by operation of the air suction fan device 100 is uniform, and in the range in which a speed pattern of the air flowing through the plurality of air suction through holes and a speed pattern when the air flows in the common suction space satisfy the similar requirements, the plurality of suction ports may be disposed in various manners, and the plurality of combined sampling/diagnosis kits may be coupled to the sampling device in various manner.

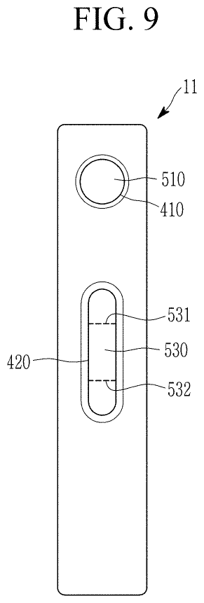

FIG. 9 is a diagram showing various arrays of the plurality of combined sampling/diagnosis kits in the common suction space.

For convenience of description, FIG. 9 shows an array of a bounding circle 204 on the common suction space 203 and the plurality of combined sampling/diagnosis kits when viewed along the z axis of FIG. 1.

In FIG. 9(a), the four combined sampling/diagnosis kits are coupled to the sampling device at a right angle in the middle of the bounding circle 204 so that the four combined sampling/diagnosis kits are kept in close touch with each other, and the sampling pads of the four combined sampling/diagnosis kits are positioned to surround the center of the bounding circle 204.

In FIG. 9(b), the four combined sampling/diagnosis kits are coupled to the sampling device so that the four combined sampling/diagnosis kits form a right angle with each other in a tangential direction of the bounding circle 204, and the sampling pads of the four combined sampling/diagnosis kits are positioned uniformly inside the bounding circle 204.

In FIG. 9(c), the eight combined sampling/diagnosis kits are coupled to the sampling device towards the center of the bounding circle 204 so that the eight combined sampling/diagnosis kits are kept in close touch in the bounding circle 204, and the sampling pads are positioned in a circular shape with respect to the center of the bounding circle 204.

In FIG. 9(d), the ten combined sampling/diagnosis kits are coupled to the sampling device in a symmetric shape with respect to the center line passing through the center of the bounding circle 204, and one half and the other half of the sampling pads of the ten combined sampling/diagnosis kits are positioned symmetrical to each other with respect to the center line.

In FIG. 9(e), the eight combined sampling/diagnosis kits are bundled up into two groups, and coupled to the sampling device at a right angle so that the eight combined sampling/diagnosis kits are kept in close touch with each other in the center of the bounding circle 204, and the sampling pads of the eight combined sampling/diagnosis kits are positioned to surround the center of the bounding circle 204.

In FIG. 9(f), the eight combined sampling/diagnosis kits are bundled up into two groups, and coupled to the sampling device so that the eight combined sampling/diagnosis kits form a right angle with each other in a tangential direction of the bounding circle 204, and the sampling pads of the eight combined sampling/diagnosis kits are positioned uniformly inside the bounding circle 204.

The positions of the plurality of suction ports are determined depending on the positions of the plurality of sampling pads disposed in FIG. 9 (a) through (f). Also, as shown in FIG. 9 (a) through (f), a structure of the air sampling device may be modified so that the plurality of combined sampling/diagnosis kits can be coupled to the air sampling device.

The air sampling drive unit 40 may include a power source 41 configured to drive the air suction fan device 100 to collect the air, a control circuit 42, and a flow rate measuring machine 43. The flow rate measuring machine 43 may measure an amount of the air discharged from the air suction fan device 100 and transmit the air into the control circuit 42, and the control circuit 42 may control the power source 43 based on the measured amount of the air. For example, to control an amount of the air collected per unit time based on predetermined conditions, the control circuit 42 regulates the output electric power of the power source 41. The control circuit 42 may control the power source 41 in a negative feedback control mechanism in which the output electric power of the power source 41 is lowered when the amount of the air is increased and the output electric power of the power source 41 is raised when the amount of the air is reduced.

The analysis device 20 may irradiate the combined sampling/diagnosis kit 35 coupled to a coupling groove 26 with infrared rays, and sense the infrared rays emitted from the test line of the kit 35 to determine whether a target material is detected and measure an amount of the detected target material, and may sense the infrared rays emitted from the control line to analyze whether the combined sampling/diagnosis kit 35 effectively performs a detection operations.

The analysis device 20 may transmit an image, which is sensed by the infrared rays emitted from the test line and the control line, to an external terminal 50. In FIG. 2, a cable 51 is shown to be connected between the analysis device 20 and the external terminal 50, but the image may be transmitted from the analysis device 20 to the external terminal 50 via wireless communications. In this case, an application capable of displaying an image taken by an infrared camera of the analysis device 20 may be installed in the external terminal 50. The external terminal may be one of various devices, such as smart phones, tablet PCs, laptop computers, and the like, which may display the received image data using the installed application.

The analysis device 20 includes a laser 21, an optical filter 22, an infrared camera 23, an image processing unit 24, and an interface 25.

The laser 21 irradiates a membrane of the combined sampling/diagnosis kit 35 with infrared rays with a certain band (e.g., 980 nm wavelength). Specifically, the laser 21 may irradiate a test line and a control line of the membrane with infrared rays with 980 nm wavelength.

The optical filter 22 may transmit wavelengths with a band less than or equal to a certain band (e.g., 850 nm). The optical filter 22 may include a visible ray cut-off filter and an ultraviolet ray cut-off filter.

The infrared camera 23 takes an image of infrared rays passing through the optical filter 22.

The image processing unit 24 processes the image taken by the infrared camera 23 to convert the image into image data, which are transmittable to the external terminal 50.

The interface 25 transmits the image data converted by the image processing unit 24 to the outside. The interface 25 may be connected to the cable 51 to transmit the image data to the external terminal 50 through the cable 51.

It is described that one of the plurality of combined sampling/diagnosis kits 31-34 coupled to the air sampling device 10 to complete the collection of the specimen is inserted into the coupling groove 26 in the analysis device 20 in order to perform the analysis, but the present invention is not limited thereto. For example, an analysis operation may be performed together in the air sampling device 10.

In the air sampling device 10, a reader module includes a laser, an optical filter, and an infrared camera, all of which have a fixed or mobile structure, may be positioned on the combined sampling/diagnosis kits 31-34. Then, after the specimen is collected in the air sampling device 10, it may be determined whether a target material is detected by irradiating a membrane of the combined sampling/diagnosis kit with infrared rays while the combined sampling/diagnosis kit is coupled to the air sampling device 10 without detachment from the air sampling device 10, and taking an image of infrared rays emitted from the combined sampling/diagnosis kit. In the case of the mobile structure, the reader module may be positioned in an empty space of the air sampling device 10 during collection of the air to prevent the reader module from interfering with collection of the air, and may move to an upper portion of the kit when the collection of the air is completed.

FIG. 9 is a diagram showing a combined sampling/diagnosis kit according to an exemplary embodiment.

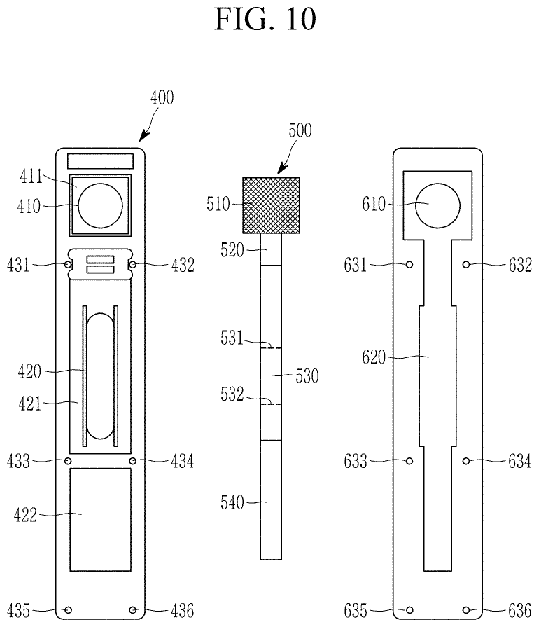

FIG. 10 is an exploded diagram showing a structure of the combined sampling/diagnosis kit according to an exemplary embodiment.

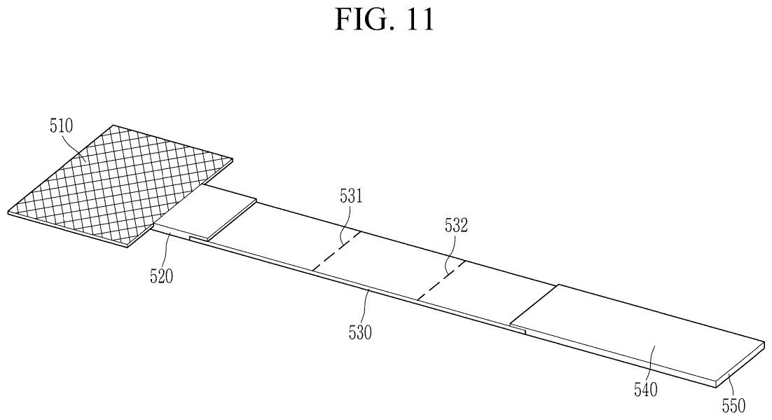

FIG. 11 is a perspective view showing a test strip according to an exemplary embodiment.

Each of the plurality of combined sampling/diagnosis kits 31-34 includes a test strip for a target material to be detected, and a plastic cartridge configured to accommodate a test strip to couple the test strip to an air sampling device. FIGS. 9 to 11 are diagrams of a combined sampling/diagnosis kit 31 which is one of the plurality of combined sampling/diagnosis kits 31-34, and may also include the same configurations of the other combined sampling/diagnosis kits 32-34.

As shown in FIG. 10, the combined sampling/diagnosis kit 31 includes an upper lid 400, a lower lid 600, and a test strip 500, which constitutes a cartridge.

The upper lid 400 includes an opening 410 configured to expose a sampling pad 510 of the test strip 500, an opening 420 configured to expose a membrane 530 of the test strip 500, accommodation spaces 411, 421, and 422 formed in an inner direction of the upper lid 400 to accommodate the test strip 500, and protrusions 431-436 for coupling to the lower lid 600. The lower lid 600 includes an opening 610 configured to expose the sampling pad 510 of the test strip 500, an accommodation space 620 formed in an inner direction of the lower lid 600 to accommodate the test strip 500, and grooves 631-636 inserted and coupled to the protrusions 431-436 for coupling to the upper lid 400.

The plurality of air suction through holes 311-314 previously shown in FIG. 4 are air suction through holes that are formed between the air suction ports 11-14 to extend from the opening 410 of the upper lid 400, and the plurality of air suction through holes 331-334 are air suction through holes that are formed to extend in a vertical direction (a normal direction to a plane shown in FIG. 10) from the opening 610 of the lower lid 600.

The opening 420 is formed in such a size that a test line 531 and a control line 532 in the membrane 530 can be exposed to the outside, and the openings 410 and 610 may be formed to expose the sampling pad 510 to the maximum extent to collect the specimen. In FIG. 10, the openings 410 and 610 are shown to be in a circular shape, but the present invention is not limited thereto. In this case, the openings 410 and 610 may be formed in quadrangular or other polygonal shapes similar to that of the sampling pad 510.

The test strip 500 may be realized to be adapted to lateral flow diagnosis, and may be manufactured using paper as a main material. The test strip 500 includes a sampling pad 510, a capture agent-nanoparticle conjugation pad 520, a membrane 530 configured to detect a signal, an absorption pad 540, and a supporter 550, and the sampling pad 510, the conjugation pad 520, the membrane 530, and the absorption pad 540 are positioned so that they are connected onto the supporter 550 in this order in a moving direction of the specimen. In a horizontal direction (a direction parallel with the moving direction of the specimen), the conjugation pad 520 is positioned in contact with the sampling pad 510, the membrane 530 is positioned in contact with the conjugation pad 520, and the absorption pad 540 is positioned in contact with the membrane 530. A portion of the sampling pad 510 may be positioned on a portion of the conjugation pad 520 so that the sampling pad 510 and the conjugation pad 520 partially overlap each other in a vertical direction (a direction vertical to the horizontal direction). A portion of the membrane 530 may be positioned between a portion of the conjugation pad 520 and the supporter 550 so that the membrane 530 and the conjugation pad 520 partially overlap each other in a vertical direction. A portion of the membrane 530 is positioned between a portion of the absorption pad 540 and the supporter 550 so that the membrane 530 and the absorption pad 540 partially overlap each other in a vertical direction.

The sampling pad 510 is formed in a porous structure, and thus may be realized using a material through which the air passes and which absorbs an analysis solution, such as glass fibers, polyester fibers, meshes, or the like. Surfaces of the glass fibers, the polyester fibers, or the meshes may be subjected to certain treatment. For example, a surface of the sampling pad 510 may be treated with a mixed solution of PVP, sucrose, BSA, and Tween 20. The sampling pad 510 may collect a specimen including a target material that is an analyte in the sucked air. The target material refers to an analyte whose concentration or presence/absence is to be analysed.

The capture agent-nanoparticle composites are dispensed in the conjugation pad 520, and the diagnostic capture agent includes an antibody, an aptamer, and the like, which specifically recognize and bind to the target material. The nanoparticles may be upconversion infrared absorption/emission nanoparticles. The infrared absorption/emission nanoparticles are doped with a rare earth element to provide upconversion nanoparticles that absorb light energy with long wavelengths and emit light energy with short wavelengths through a pyrolytic synthesis reaction. The capture agent-nanoparticle composites specifically bind to a target material, and emit infrared rays other than visible rays when the capture agent-nanoparticle composites absorb the infrared rays. The emitted infrared rays have a long wavelength, and thus may pass through a sample and do not produce background signals. Therefore, because there is no interference between light absorption and emission, a target material to be detected may be detected with high sensitivity. Upon a detection operation of the target material, the conjugation pad 520 may be pretreated with a solution obtained by mixing PVP, BSA, sucrose, Tween 20, and the like in order to efficiently recover the capture agent-nanoparticle composites from the conjugation pad 520.

When the analysis solution is dispensed in the sampling pad 510 and the specimen is allowed to move to the conjugation pad 520 together with the analysis solution, the composites in the conjugation pad 520 may be conjugated to the target material included in the specimen. The analysis solution is a solution that is dispensed in the sampling pad 510 to detect the target material and analyze an amount of the target material, and may be a buffer solution used to dissolve cells. The conjugation pad 520 may include the composites, which bind to the target material that is an analyte in the specimen, in a dried state. After the analysis solution is allowed to flow and accommodated in the conjugation pad 520, a capture agent in the composites specifically binds to the target material when the target material is included in the specimen.

The capture agent-nanoparticle composites absorb infrared rays to emit infrared rays when irradiated with the infrared rays. In this case, the wavelength of the absorbed infrared rays is not identical to the wavelength of the emitted infrared rays. For example, the capture agent-nanoparticle composites may absorb infrared rays with long wavelengths to emit infrared rays with short wavelengths, wherein the infrared rays with long wavelengths may be infrared rays having a wavelength of 960 to 980 nm, and the infrared rays with short wavelengths may be infrared rays having a wavelength of 750 to 850 nm. The infrared rays having a wavelength of 750 to 850 nm may have enhanced transmittance to bio-materials such as tissues, and the like, thereby preventing an effect by the specimen such as blood, feces, and the like. Also, because the infrared rays have a high transmittance to an opaque mixed solution of the specimen, a target material may be detected from various types of collected specimens. Also, because the infrared rays having a wavelength of 750 to 850 nm do not produce autofluorescence, a signal to noise ratio may be improved. The combined sampling/diagnosis kit according to an exemplary embodiment may solve the problems such as low sensitivity while maintaining convenience and economic feasibility of conventional immunoassay diagnosis kits used in the fields.

When the nanoparticle is further doped with a heterogeneous dopant, distortion of a crystal structure in the nanoparticles may be increased to some extent, thereby enabling the transfer of very sensitive electrons. In this way, the luminance intensity may be further enhanced without any change in size of the nanoparticles themselves.

The nanoparticles according to an exemplary embodiment may include any one or more selected from the group consisting of a fluoride, an oxide, a halide, an oxysulfide, a phosphate, and a vanadate. For example, the nanoparticles may include any one or more selected from the group consisting of NaYF4, NaYbF4, NaGdF4, NaLaF4, LaF3, GdF3, GdOF, La2O3, Lu2O3, Y2O3, and Y2O2S. The rare earth element with which the nanoparticles are doped may include a lanthanide element, and a range of wavelengths of light in which the nanoparticles absorb and emit light may be regulated by regulating the type and concentration of the rare earth element included in the nanoparticles. The nanoparticles having no interference in a range of wavelengths absorbing and emitting the infrared wavelengths may be provided by regulating the type and concentration of the rare earth element. For example, the rare earth element may include any one or more selected from the group consisting of Y, Er, Yb, Tm, and Nd. More specifically, the rare earth element may include 45 to 55 mol % of Y, 43 to 52 mol % of Yb, and 1.5 to 3 mol % of Tm.

The luminance intensity of the nanoparticles may be regulated by regulating the type or concentration of the heterogeneous dopant. Examples of the heterogeneous dopant with which the nanoparticles are further doped may include any one or more selected from the group consisting of Ca, Si, Ni, and Ti.

The nanoparticles doped with the rare earth element and the heterogeneous dopant may be manufactured by doping according to the methods commonly used in the technical field of the present invention, and may, for example, be manufactured using a method disclosed in Qian et al., Small, 5: 2285-2290, 2009; Li et al., Advanced Materials, 20: 4765-4769, 2008; Zhao et al., Nanoscale, 5:944-952, 2013; Li et al., Nanotechnology, 19: 345606, 2008. The above-mentioned documents may be incorporated herein by reference in their entireties.

A bond of the diagnostic capture agent (composed of an antibody, an aptamer, and the like) to the nanoparticles doped with the rare earth element and the heterogeneous dopant includes bonds selected from an ionic bond, a covalent bond, a metallic bond, a coordinate bond, a hydrogen bond, and a Van der Waals bond.

The nanoparticles may include a core layer, a shell layer, and a coating layer. The core layer may be composed of particles doped with a rare earth element. The shell layer is further doped with a heterogeneous dopant to surround the core layer, thereby reducing surface defects and improving surface uniformity. The coating layer is formed by coating an outer surface of a cell layer with a monomer or a polymer, thereby enhancing dispersibility of the nanoparticle in a fluid and facilitating fixation of the diagnostic capture agent. The diagnostic capture agent binds to the coating layer. When the nanoparticles have a core-shell structure, surface defects may be reduced to enhance surface uniformity, and monodispersity may be enhanced to maximize infrared emission efficiency. When the shell layer is further doped with the heterogeneous dopant, infrared luminance intensity may be further improved. When the nanoparticles are surface-treated with a monomer or a polymer, the capture agent-nanoparticle composites may have enhanced dispersibility in an analysis solution and fixation of the antibody in the capture agent-nanoparticle composites may be facilitated.

For example, the core layer is formed in the form of nanoparticles by mixing 1-octadecene, oleic acid, and a rare earth element to form a homogeneous solution, mixing methanol containing sodium hydroxide and fluorine ammonium with the homogeneous solution, agitating the resulting mixture, and then reacting the mixture at a constant temperature for a constant time, and the shell layer is formed on the core layer to have a constant thickness by mixing 1-octadecene, oleic acid, a rare earth element, and a heterogeneous dopant to form a homogeneous solution, mixing methanol containing sodium hydroxide and fluorine ammonium with the homogeneous solution together with the core layer, agitating the resulting mixture, and then reacting the mixture at a constant temperature for a constant time.

The polymer used to form a coating layer may include one or more selected from the group consisting of polyacrylic acid (PAA), polyallylamine (PAAM), 2-aminoethyl dihydrogen phosphate (AEP), polyethylene glycol diacid, polyethylene glycol acid, and polyethylene glycol phosphate ester. The formation of the coating layer may be performed using methods commonly taken in practice in the art. For example, the coating layer may be treated by means of ligand engineering such as ligand exchange or oleic acid oxidation, ligand attraction, layer-by-layer assembly, surface treatment using silanization, surface polymerization, and the like. Also, the coating layer may be surface-treated according to the method disclosed in Photon Upconversion Nanomaterials, Fan Zhang, Springer, 2015, the contents of which may be incorporated herein by reference.

A test line 531 capable of detecting a target material, and a control line 532 capable of determining whether the test strip 500 is normally operated are present in the membrane 530, a capture antibody capable of binding to the target material is fixed onto a membrane 530 in the test line 531, and a control antibody binding to the capture agent-nanoparticle composites is fixed onto the membrane 530 in the control line 532. The test line 531 may be positioned in closer proximity to the conjugation pad 520 than the control line 532.

The capture antibody of the test line 531 is configured to specifically bind to or react with the target material. For example, an antibody, an aptamer, and the like may be used. The control antibody is configured to specifically bind to or react with the capture agent of the composites. For example, an antibody, an aptamer, and the like may be used.

The target material specifically binding to the capture agent in the composites may be allowed to move from the conjugation pad 520 to the membrane 530 so that a portion of the target material may be fixed in the test line 531 by binding to the capture antibody, and a portion of the target material may be fixed in the control line 532 by allowing the capture agent in the composites to react with the control antibody.

The capture antibody reacting with the target material is fixed in the test line 531.

It may be determined whether the target material to be analyzed is included in the collected specimen, and a concentration of the target material may be analyzed by determining whether the infrared rays are emitted from the test line 531, and measuring the luminance intensity.

Because the control antibody reacting with the capture agent in the composites is fixed in the control line 532, it may be judged whether the specimen moves to a position necessary for detection of the target material and whether the capture agent in the composites is operated by determining whether the infrared rays are emitted from the control line 532. Therefore, these results may be used as the criteria for reading validity and effectiveness of analysis.

The absorption pad 540 is a pad that absorbs a fluid in the specimen passing through the membrane 530. The absorption pad 540 may serve as a pump that absorbs the dispensed analysis solution and allows the specimen to continuously move from the sampling pad 510 to the membrane 530. The analysis solution may, for example, be a solution including any one or more selected from the group consisting of phosphate buffer saline (PBS), KCl, NaCl, Tween20, 4-(2-hydroxyethyl)-1-piperazineethanesulfonic acid (HEPES), NaN3, and the like, but the present invention is not limited thereto.

The conjugation pad 520, the membrane 530, and the absorption pad 540 may include a solid-phase capillary support, and the solid-phase capillary support may be used without limitation as long as it is a porous polymer that may serve as a solid-phase capillary carrier for a chemical components such as antigens, antibodies, aptamers, or haptens, or is a natural or synthetic material, or a naturally occurring material modified by synthesis. In this case, a shape of the solid-phase capillary support is not limited. As described above, for example, the solid-phase capillary support may include one or more selected from the group consisting of a cellulose material, paper, cellulose acetate, nitrocellulose, polyether sulfone, polyethylene, nylon, polyvinylidene fluoride (PVDF), polyester, polypropylene, silica, vinyl chloride, a vinylchloride-propylene copolymer and a vinyl chloride-vinyl acetate copolymer, inactivated alumina, diatomite, MgSO4, cotton, nylon, rayon, silica gel, agarose, dextran, gelatin, and polyacrylamide. As a more specific example, the membrane may include at least one polymer selected from the group consisting of nitrocellulose, polyether sulfone, polyethylene, nylon, polyvinylidene fluoride, polyester, and polypropylene. As an exemplary embodiment, the solid-phase capillary support may also have a shape like a rod, a plate, a tube, a bead, or the like.

The porosity of the sampling pad 510 is very greater than the porosities of the conjugation pad 520, the membrane 530, and the solid-phase capillary support of the absorption pad 540. That is, the sampling pad 510 has such a porosity that the sucked air can pass through the sampling pad 510, and the conjugation pad 520, the membrane 530, and the solid-phase capillary support of the absorption pad 540 have such a porosity that the chemical components can move by means of a capillary phenomenon.

All type of the supporter 550 may be used without limitation as long as they can support and carry the sampling pad 510, the conjugation pad 520, the membrane 530, and the absorption pad 540. In this case, the supporter 550 may be impermeable to a liquid to prevent the specimen from leaking from the analysis solution. For example, the supporter 550 may include glass, polystyrene, polypropylene, polyester, polybutadiene, polyvinyl chloride, polyamide, polycarbonate, epoxide, methacrylate, polymelamine, and the like.

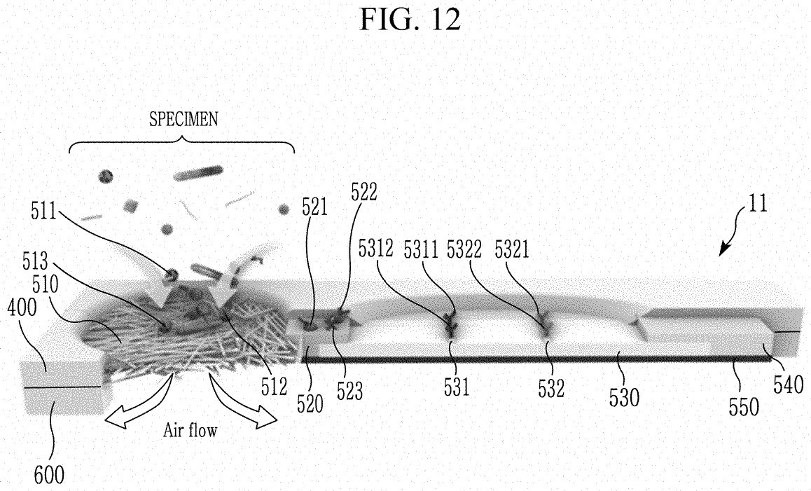

Hereinafter, an operation of the combined sampling/diagnosis kit will be described with reference to FIGS. 12 to 14.

FIGS. 12 to 14 are perspective cross-sectional view of a combined sampling/diagnosis kit for explaining an operation of the combined sampling/diagnosis kit.

FIGS. 12 to 14 are diagrams showing a combined sampling/diagnosis kit 31 that is one of four combined sampling/diagnosis kits 31-34.

FIG. 12 is a diagram schematically showing an operation of the combined sampling/diagnosis kit to collect a specimen.

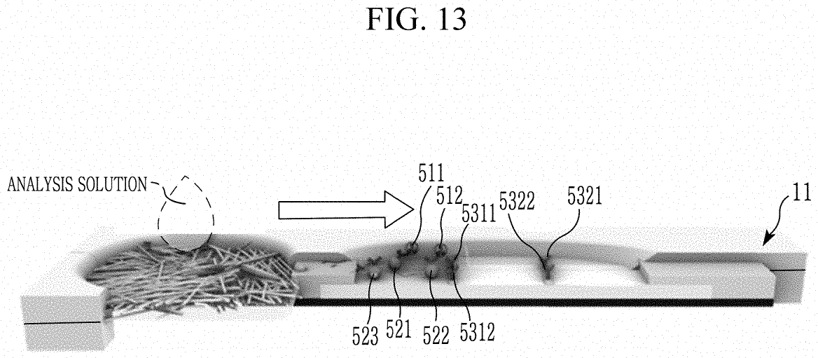

FIG. 13 is a diagram schematically showing movement of the specimen by an analysis solution after the analysis solution is dispensed in the combined sampling/diagnosis kit.

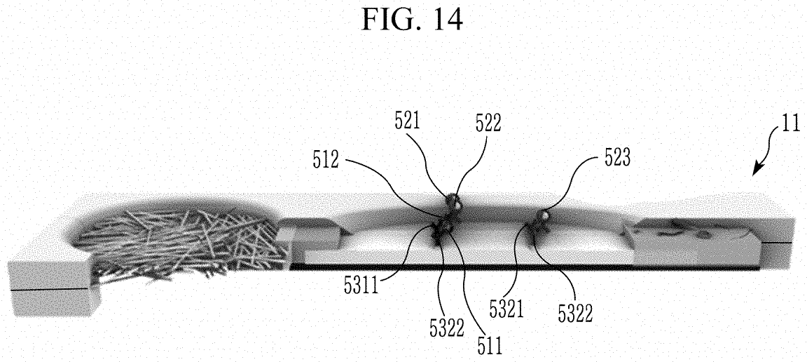

FIG. 14 is a diagram schematically showing a state of a test line and a control line of the combined sampling/diagnosis kit after the combined sampling/diagnosis kit absorbs the analysis solution.

As shown in FIG. 12, the air is sucked through the sampling pad 510 positioned at one end of the combined sampling/diagnosis kit 31 to collect a specimen, which includes various materials including pathogens in the air, in the sampling pad 510. In FIG. 12, it is assumed that target materials 511-513 are included in the specimen for convenience of description. In addition, to explain a process of binding the target material to the composites and the capture antibody and a process of binding the composites to the control antibody, three composites 521-523, two capture antibodies 5311, and 5312, and two control antibodies 5321, and 5322 are shown on the conjugation pad 520, the test line 531, and the control line 532, respectively.

As shown in FIG. 13, when the analysis solution is dispensed after collection, cells included in the specimen are dissolved in a solution so that the cells flows from the sampling pad 510 towards the absorption pad 540 by a capillary action. In this case, the analysis solution may be a lateral flow stream-induced and cytolytic solution (a loading/lysis buffer). Then, the capture agent-nanoparticle composites 521-523 fixed in the conjugation pad 520 may react with the target materials 511-513 to form secondary composites (composites formed by an antigen-antibody reaction). For example, the target material 511 may react with the composite 521 to form a secondary composite 511+521, and the target material 512 may react with the composite 522 to form a secondary composite 512+522. In FIG. 13, it is shown that two of the three target materials 511-513 react with the composites 521 and 522. This is one example intended to describe that at least a portion of the target material collected in the sampling pad 510 reacts with a portion of the plurality of composites, but the present invention is not limited thereto.

The secondary composites 511+521 and 512+522 move to the membrane 530 along a lateral flow stream, and bind to the capture antibodies 5311 and 5312 fixed in the test line 531 of the membrane 530. The target materials (antigens) 511 and 512 of the secondary composites 511+521 and 512+522 are bound to the capture antibodies 5311 and 5312 on the test line 531 by means of an antigen-antibody reaction, and fixed in the test line 531.

As shown in FIG. 14, the composite 523 which does not react with the target material reacts with the control antibody 5322 fixed in the control line 532 so that the composite 523 is fixed in the control line 532. When the composite does not include the target materials (antigens), the antigen-antibody reaction does not occur in the test line 531. As a result, the composite 523 is allowed to move to the control line 532 to bind to the control antibody 5322.

When amounts of the capture agent-nanoparticle composites fixed in the test line 531 and the control line 532 are analyzed using optical or chemical signals, and the like, it may be determined whether the target material is detected in the specimen, and an amount of the detected target material may be measured. In fact, a detection operation in the test line 531 and the control line 532 may be performed within 20 minutes.

An immunoassay-based detection process according to an exemplary embodiment may proceed without detaching the combined sampling/diagnosis kits from the microbial sensor device. There is no effect on the results even when the detection process proceeds in separate manner. Because the collection and detection processes are directly performed without any relocation or time delay, the activity of the collected microorganisms may be maintained. Therefore, there is no need for use of additional buffers and containers or certain sampling conditions for this purpose.

Hereinafter, the implementability and effect of the air sampling device according to an exemplary embodiment will be described by describing Experimental Examples.

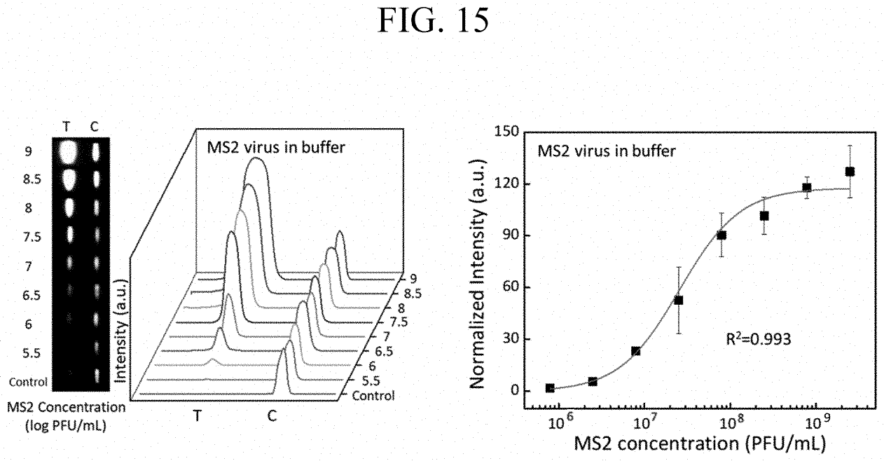

FIG. 15 is a graph showing the results of detection of MS2 viruses using the combined sampling/diagnosis kit coupled to the air sampling device.

The MS2 viruses present in a solution for an experiment may be detected by the combined sampling/diagnosis kit when a concentration of the MS2 viruses is greater than or equal to 10⁶ PFU/mL, indicating that the detection limit of the combined sampling/diagnosis kit is ten-fold lower than that of an enzyme-linked immunosorbent assay (ELISA). For an experiment, a high concentration (10¹¹ PFU (plaque forming unit)/mL) of a virus stock solution was diluted with a cytolytic buffer to prepare various concentrations (e.g., 10⁹ to 10^{5.5} PFU/mL) of an experimental solution including the viruses, and 100 uL of the experimental solution was dispensed in a sampling pad. The concentration is in a range of 10⁸ to 10^{4.5} PFU/100 uL based on the total amount of the analysis solution. A PBS buffer diluted under the same conditions was used for an ELISA method. In the ELISA method, the viruses were detected after the concentration of the viruses is greater than 10⁷ PFU/mL.

As shown in FIG. 15, it can be seen that, as the concentration of the viruses exceeds 10⁶ PFU/mL, the emission of infrared rays was detected in the test line T, the intensity of the infrared rays emitted from the test line T increased as the concentration of the viruses increased. In this case, it can be seen that the combined sampling/diagnosis kit is normally operated as the infrared rays emitted from the control line C are detected.

In the experiment, a protein, a surfactant, or the like may be used to treat a surface of the sampling pad. For example, a surface of the sampling pad may be treated with a single solution or a mixed solution, which contains approximately 0.1 to 5% of polyvinylpyrrolidone (PVP), sucrose, bovine serum albumin (BSA), Tween 20, and the like.

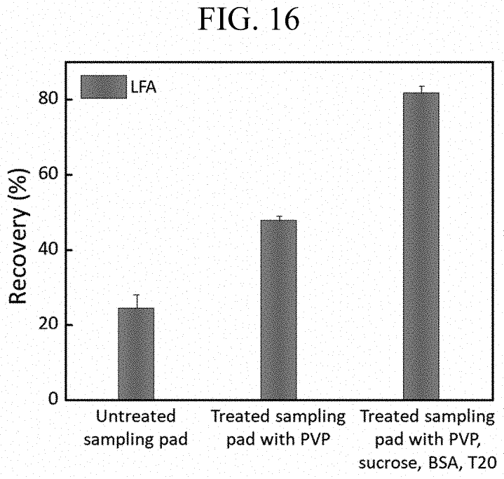

FIG. 16 is a graph showing a recovery rate of MS2 viruses.

As shown in FIG. 16, it can be seen that the recovery rate (47.7%) when a surface of the sampling pad is treated with a PVP solution (a PVP-treated sampling pad), and the recovery rate (81.7%) when a surface of the sampling pad is treated with a mixed solution of PVP, sucrose, BSA, and Tween 20 (a PVP/sucrose/BSA/Tween 20-treated sampling pad) is higher compared with the recovery rate (24.3%) when the sampling pad is not subjected to surface treatment (an untreated sampling pad). The recovery rate refers to a ratio of an amount of the MS2 viruses separated from the sampling pad with movement of the analysis solution to a total amount of the MS2 viruses collected in the sampling pad.

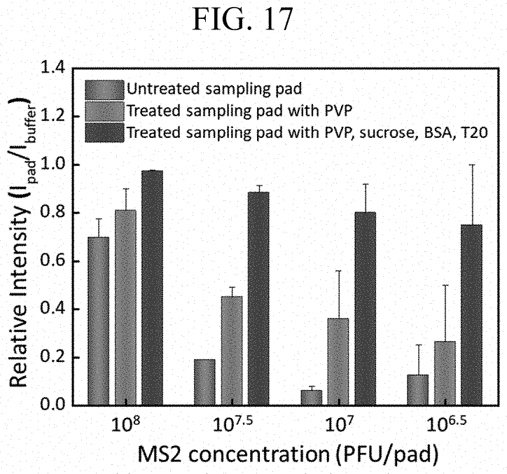

FIG. 17 is a graph showing a detection ratio of the MS2 viruses recovered from the sampling pad.

As shown in FIG. 17, the detection ratio is a relative intensity, and may be quantified by Ipad/Ibuffer. The Ibuffer is an intensity of infrared rays emitted from the nanoparticles observed relative to the amount of the MS2 viruses detected on the membrane when the viruses are diluted with a buffer solution and dispensed in the membrane. The Ipad is an intensity of infrared rays emitted from the nanoparticles observed relative to the amount of the MS2 viruses detected on the membrane when a buffer solution is dispensed in the detection pad after the same amount of the viruses are dried in the detection pad. That is, the Ipad is an intensity of the infrared rays emitted from the nanoparticles when processes of collecting floating viruses in the pad and drying the floating viruses are reproduced. When the relative intensity calculated by the Ipad/Ibuffer becomes closer to 1, it can be observed that the viruses adsorbed onto the sampling pad are easily re-dispersed in an elution and buffer solution, and then move towards the test line of the membrane.

FIG. 17 shows the detection ratios when using the sampling pad not subjected to surface treatment (an untreated sampling pad), the sampling pad whose surface is treated with a varying concentration of a PVP solution (a PVP-treated sampling pad), and the sampling pad whose surface is treated with a varying concentration of a mixed solution of PVP, sucrose, BSA, and Tween 20 (a PVP/sucrose/BSA/Tween 20-treated sampling pad) while varying the concentration of the MS viruses.

As shown in FIG. 17, the detection ratio is the lowest when the untreated sampling pad is used, the detection ratio is the second highest when the sampling pad whose surface is treated with a PVP solution is used, and the detection ratio is the highest when the sampling pad whose surface is treated with a mixed solution of PVP, sucrose, BSA, and Tween 20 is used.

The higher detection ratio means the higher probability that the MS2 viruses recovered from the sampling pad bind to the capture agent-nanoparticle composites in the conjugation pad, move to the membrane, and bind to the test line. Then, when the MS2 viruses are present at the same concentration in the sampling pad, the MS2 viruses bind to the test line with the highest recovery rate in the pad treated with the mixed solution of PVP, sucrose, BSA, and Tween 20, resulting in the highest signaling reaction of the nanoparticles. Sensitivities of signals may be improved with an increasing signaling reaction of the nanoparticles.

Hereinafter, a chamber for an experiment used to collect air in the atmosphere and detect a target material will be described.

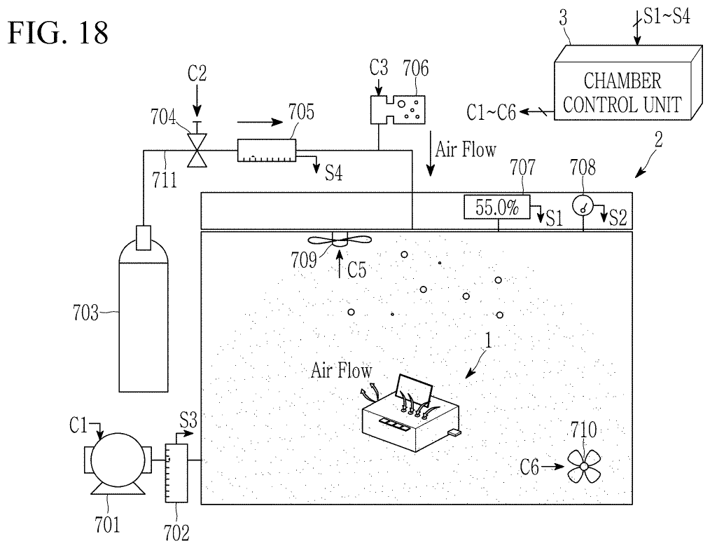

FIG. 18 is a diagram schematically showing a chamber for an experiment used to collect air and detect a target material.

As shown in FIG. 18, a chamber 2 includes a pump 701, flow rate measuring systems 702 and 705, an air cylinder 703, a valve 704, a sprayer (a collision nebulizer) 706, a hygrometer 707, a thermometer 708, and two fans 709 and 710.

A chamber control system 3 may be connected to individual configurations of the chamber 2 via wire or wireless communications to receive detection signals S1-S4, and produce and transmit control signals C1-C6 in consideration of the experimental conditions.

The pump 701 supplies external air into the inside of the chamber 2 according to the control signal C1 received from the chamber control system 3. The flow rate measuring system 702 may measure an amount of the air supplied from the pump 701 into the inside of the chamber 2 to produce a detection signal S3 and transmit the detection signal S3 to the chamber control system 3.

The air cylinder 703 stores the air required to produce a bioaerosol. The valve 704 controls an amount of the air supplied through a piping 711 according to the control signal C2 received from the chamber control system 3. The flow rate measuring system 705 may measure an amount of the air supplied through the piping 711 to produce a detection signal S4 and transmit the detection signal S4 to the chamber control system 3.

The sprayer 706 mixes a microbial solution with the air supplied through the piping 711 and sprays the mixture into the chamber 2 according to the control signal C3 received from the chamber control system 3. Then, the aerosol including the microorganisms flows in the chamber 2.

The hygrometer 707 may measure humidity in the chamber 2 to produce a detection signal S1 and transmit the detection signal S1 to the chamber control system 3, and the thermometer 708 may measure a temperature in the chamber 2 to produce a detection signal S2 and transmit the detection signal S2 to the chamber control system 3.

Each of the two fans 709 and 710 operates according to the control signals C5 and C6 to control the flow of the air in the chamber 2.

As such, the bioaerosol may be artificially produced in the chamber 2, and the surrounding environments such as a temperature, humidity, a pressure, and the like may be promoted according to the experimental conditions. In the promoted experimental conditions, the air sampling device 1 may be operated to perform an experiment for detection of a target material using the combined sampling/diagnosis kit coupled to the air sampling device 1.

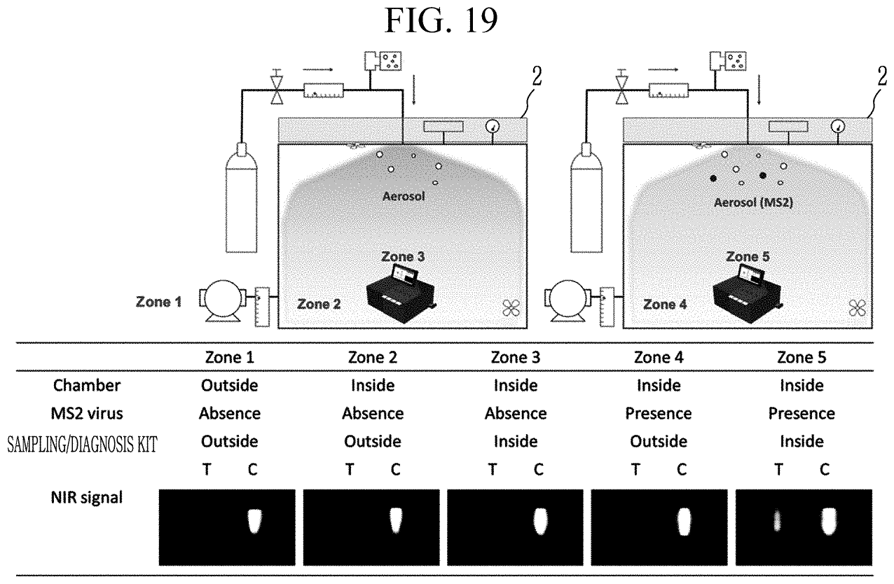

FIG. 19 is a diagram showing the results of evaluating the collection performance of the microbial sensor device to which the combined sampling/diagnosis kit is coupled.

Zone 1 is the outside of the chamber 2, and Zones 2-5 are the inside of the chamber 2.

In this case, as shown in FIG. 19, the MS2 viruses were not detected in Zones 1-3 under an MS2 virus-free environment (Absence). That is, the infrared rays were emitted from the control line C only, but the infrared rays were not emitted from the test line T.

In Zone 4 with an environment in which the MS2 viruses are present, when the combined sampling/diagnosis kits are positioned outside the air sampling device 10 (Outside), the air sampling device 10 collects a specimen, but does not detect the MS2 viruses.

In Zone 5 with an environment in which the MS2 viruses are present, when the combined sampling/diagnosis kits are coupled to the inside of the air sampling device 10 (Inside), the air sampling device 10 collects a specimen. As a result, it can be seen that the infrared rays are emitted from the test line T of the combined sampling/diagnosis kit to detect the MS2 viruses.

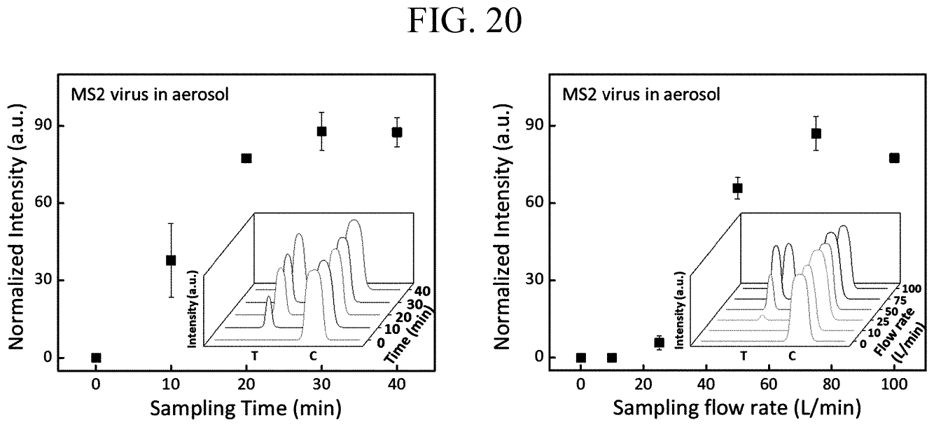

FIG. 20 is a graph showing the results of detection of MS2 viruses depending on a sampling time and a flow rate in the microbial sensor device.

As shown in FIG. 20, it can be seen that the luminance intensity of the infrared rays from the test line T increases with the elapse of a sampling time, and then decreases after the elapse of approximately 30 minutes. Based on these results, it is possible to set the sampling time of the microbial sensor device 1. Also, when a sampling flow rate is greater than or equal to approximately 25 L/min, the infrared rays start to emit at the test line T. Based on these results, it is possible to set an air sampling flow rate of the microbial sensor device 1.

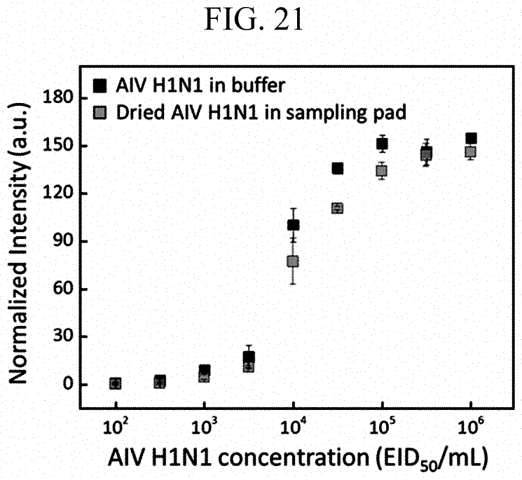

FIG. 21 is a graph showing the results of detection of avian influenza virus using the microbial sensor device.

When the capture agent-nanoparticle composites positioned on the conjugation pads of the combined sampling/diagnosis kits are prepared, a diagnostic capture agent that specifically recognizes and binds to avian influenza virus (AIV H1N1) was used. It is revealed that the avian influenza virus present in an aqueous solution (a buffer) (AIV H1N1 in a buffer) may be detected from 10³ 50% egg infectious dose (EID₅₀/mL), and the avian influenza virus adsorbed in the sampling pad (dried AIV H1N1 in the sampling pad) may be detected from 10^{3.5} EID₅₀/mL.

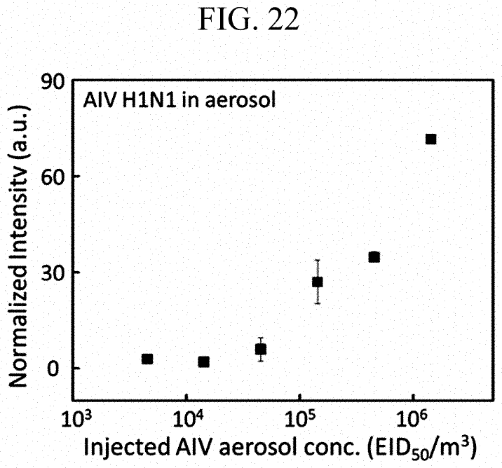

FIG. 22 shows the results of collection and detection of avian influenza virus in the air using the microbial sensor device.

As shown in FIG. 22, it can be seen that the detection intensity increases with an increasing aerosol concentration of the avian influenza virus. The avian influenza virus in an aerosol phase sprayed in the air (AIV H1N1 in aerosol) has a level of detection intensity detectable from 10^{5.5} (EID₅₀/m³).

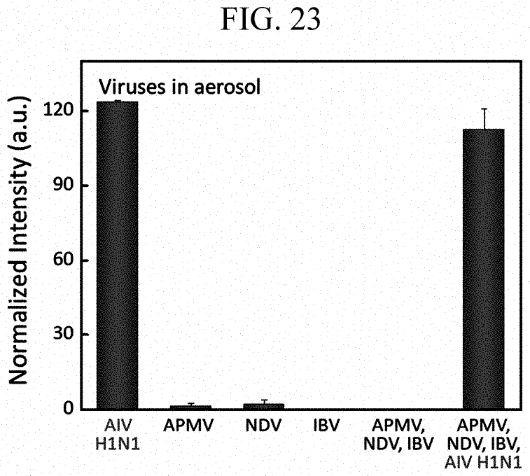

FIG. 23 shows the results showing diagnostic selectivity to the avian influenza virus in the microbial sensor device.

It is revealed that the combined sampling/diagnosis kits manufactured using the diagnostic capture agent, which specifically recognizes and reacts with the avian influenza virus (AIV H1N1) even when non-targeted avian infection-related viruses (avian paramyxovirus (APMV), Newcastle disease virus (NDV), infectious bronchitis virus (IBV)) are present, may selectively detect the avian influenza virus (AIV H1N1) only.

While the microbial sensor device collects an atmospheric sample, a combined collection/detection sensor platform may use a disposable cover to minimize contamination of a surface of equipment and user's infection. The disposable cover is sized to cover a surface of the microbial sensor device except for a suction port through which an atmospheric specimen is sucked, and is allowed to be easily attachable and detachable to the microbial sensor device using a fastening clip, and the like. Also, the disposable cover may be made of materials that filter off external dust and contaminants, such as vinyl, paper, and the like.

—Preparation of Infrared Absorption/Emission Nanoparticles—

(1) Formation of Core

1-octadecene, oleic acid, yttrium acetate hydrate, ytterbium acetate hydrate, and thulium acetate hydrate were mixed (specifically, 0.4 mmol of lanthanides (composed of 50 mol % Y, 48 mol % Yb, and 2 mol % Tm) were mixed with 7 mL of 1-octadecene and 3 mL of oleic acid)), and then heated to 150° C. to form a homogeneous solution, and the homogeneous solution was cooled to 50° C.

5 mL of methanol containing 1 mmol NaOH and 1.6 mmol NH₄F was added to the homogeneous solution, and agitated for an hour to form a mixed solution. To remove the methanol, the mixed solution was maintained at 100° C. for 10 minutes, and maintained for 30 minutes under a vacuum state, and then maintained at 290° C. for an hour and 30 minutes under an argon gas.

The nanoparticles after the mixed solution was cooled naturally were precipitated with ethanol, and washed three times with cyclohexane and ethanol to obtain nanoparticles (a core).

(2) Formation of Shell (Formation of UCNPs)

1-octadecene, oleic acid, yttrium acetate hydrate, and calcium acetate hydrate were mixed (specifically, 0.2 mmol of a dopant (a lanthanide (Y) was mixed with 7 mL of 1-octadecene and 3 mL of oleic acid), and then heated to 150° C. to form a homogeneous solution, and the homogeneous solution was cooled to 50° C.

The nanoparticles (a core) prepared in the section “(1) Formation of core” were mixed, and heated to 100° C. to remove the cyclohexane, and then cooled again to 50° C. 5 mL of methanol containing 1 mmol NaOH and 1.6 mmol NH₄F, and the homogeneous solution were mixed, and agitated for 30 minutes. To remove the methanol, the mixed solution was maintained at 100° C. for 10 minutes, maintained for 30 minutes under a vacuum state, and then maintained at 290° C. for an hour and 30 minutes under an argon gas. The nanoparticles after the mixed solution was cooled naturally were precipitated with ethanol, and washed three times with cyclohexane and ethanol to obtain nanoparticles having a core-shell structure (Core/Shell, UCNPs)

—Preparation of Capture Agent (Antibody)-Nanoparticle Composites—

(1) Formation of Coating Layer

The nanoparticles (core/shell) were coated with a polymer using a ligand engineering method. The nanoparticles prepared in the section “(2) of Preparation of infrared absorption/emission nanoparticles” were dispersed in 13.4 mL of tetrahydrofuran to prepare a nanoparticle solution, 100 mg of dopamine hydrochloride was dispersed in 600 uL of distilled water, and then added to the nanoparticle solution to form a mixed nanoparticle solution, and the mixed nanoparticle solution was then maintained at 50° C. for 5 hours under an argon gas. The mixed nanoparticle solution was cooled naturally, and 16 uL of hydrochloric acid was then added. Thereafter, the resulting mixture was washed twice with distilled water to obtain nanoparticles (NH₂-UCNPs) having an amine group.

(2) Antibody Binding (Formation of Antibody-Nanoparticle Composites)

First, 62 ug of a monoclonal anti-MS2 bacteriophage antibody (a first antibody for capturing MS2 viruses) or a monoclonal anti-AIV nucleoprotein antibody (a first antibody for capturing a nucleoprotein of avian influenza virus) was added to 1 uL of a solution formed by mixing 1.0 mg of N-succinimidyl-S-acetyl-thioacetate (SATA), 86 uL of dimethyl sulfoxide, and 611 uL of 10 mM 4-(2-hydroxyethyl)-1-piperazineethanesulfonic acid (HEPES), and reacted at room temperature for 30 minutes, and 1 uL of a solution of 0.5 M hydroxylamine hydrochloride was added, and reacted for another 2 hours. Thereafter, the materials remaining after the reaction were removed using a 30 k filter tube to obtain a thiolated antibody. 0.25 mg of the nanoparticle having an amine group prepared in the section “Preparation of capture agent (antibody)-nanoparticle composites”, 291 uL of distilled water, and 3.7 uL of a 10 mM HEPES buffer solution were mixed to prepare a first solution, and 2.5 mg of sulfosuccinimidyl4-(N-maleimidomethyl)cyclohexane-1-carboxylate (Sulfo-SMCC) was added to 100 uL of a 10 mM HEPES buffer solution to prepare a second solution. 1 uL of the second solution was mixed with the first solution, and reacted for 2 hours. Thereafter, the materials remaining after the reaction were removed using a 30 k filter tube to obtain maleimided nanoparticles. The thiolated antibody and the maleimided nanoparticles were added to a HEPES buffer solution, reacted at 4° C. for 24 hours, and then centrifuged to obtain antibody-fixed nanoparticles (antibody-nanoparticle composites).

—Preparation of Diagnosis Kit Using Antibody-Nanoparticle Composites—

A sampling pad was fully immersed in a 10 mM HEPES buffer solution containing 0.3% (w/v) polyvinylpyrrolidone (PVP) or a 10 mM HEPES buffer solution containing 0.3% (w/v) PVP, 2.0% (w/v) bovine serum albumin (BSA), 2.0% (w/v) Tween 20, and 2.5% (w/v) sucrose, dried completely, and cut into pieces with a size of 8 mm×10 mm. The absorption pad was used after moisture was removed. A nitrocellulose membrane was laminated on a plastic card (a supporter) using a laminator, and a second antibody (A polyclonal anti-MS2 antibody in the case of MS2 viruses, and an anti-nucleoprotein antibody having an epitope different from the first antibody in the case of avian influenza virus), which reacted with an antigen included in the specimen, and a third antibody (an anti-rabbit antibody in the case of MS2 viruses, and an anti-mouse antibody in the case of avian influenza virus), which reacted with the first antibody fixed in the antibody-nanoparticle composites, were dispensed in regions of the test line T and the control line C, respectively, using an automatic dispenser, and then dried at room temperature for 48 hours. The conjugation pad was fully immersed in a 10 mM HEPES buffer solution containing 2.0% (w/v) bovine serum albumin (BSA), 2.0% (w/v) Tween 20, 2.5% (w/v) sucrose, and 0.3% (w/v) PVP, and dried using a dryer, and the prepared solution of antibody-nanoparticle composites was dispensed, completely dried in a dryer, and then used.

(2) The sampling pad, the conjugation pad, the membrane positioned on the supporter, and the absorption pad prepared above were overlapped and fixed, as shown in FIG. 11. Thereafter, a plastic case that was open to allow the air to flow therethrough was inserted into a portion of the sampling pad to prepare a combined sampling/detection kit.

Although exemplary embodiments of the present invention have been described above in detail, the exemplary embodiments are not intended to limit the scope of the present invention, and various changes and modifications made by those skilled in the art to which the present invention pertains also fall within the scope of the present invention.

### DESCRIPTION OF SYMBOLS

1: microbial sensor device
10: air sampling device
20: analysis device
30: combined sampling/diagnosis kit
40: air sampling drive unit
100: air suction fan device
200: hollow air suction device
300: air suction port device

What is claimed is:

1. A test strip comprising:
a porous sampling pad through which the air passes to collect a specimen;
a conjugation pad which is positioned on a supporter in contact with the sampling pad, and in which a plurality of capture agent-nanoparticle composites specifically binding to a target material are dispensed;
a membrane which is positioned on the supporter in contact with the conjugation pad, and comprises a test line to which the capture agent-nanoparticle composites to which the target material binds are conjugated when the specimen moves and a control line to which the capture agent-nanoparticle composites are conjugated; and
an absorption pad configured to absorb the moving specimen on the supporter.

2. The test strip of claim 1, wherein:
the sampling pad, the conjugation pad, the membrane, and the absorption pad are positioned to be connected in this order along a moving direction of the specimen.

3. The test strip of claim 2, wherein:
a portion of the sampling pad is positioned on a portion of the conjugation pad so that the sampling pad and the conjugation pad partially overlap each other in a direction vertical to the moving direction.

4. The test strip of claim 2, wherein:
a portion of the membrane is positioned between a portion of the conjugation pad and the supporter so that the membrane and the conjugation pad partially overlap each other in a direction vertical to the moving direction.

5. The test strip of claim 2, wherein:
a portion of the membrane is positioned between a portion of the absorption pad and the supporter so that the membrane and the absorption pad partially overlap each other in a direction vertical to the moving direction.

6. The test strip of claim 1, wherein:
when an analysis solution is dispensed in the sampling pad so that the specimen moves towards the conjugation pad along with the analysis solution, the target material in the specimen binds to any one of the plurality of capture agent-nanoparticle composites.

7. The test strip of claim 6, wherein:
a plurality of capture antibodies are fixed in the test line, and one of the plurality of capture antibodies binds to the target material of the capture agent-nanoparticle composites to which the target material binds.

8. The test strip of claim 6, wherein:
a plurality of control antibodies are fixed in the control line, the control line is positioned behind the test line in a moving direction of the specimen, and one of the plurality of control antibodies binds to the capture agent of the capture agent-nanoparticle composites.

9. The test strip of claim 1, wherein:
the capture agent-nanoparticle composites absorb infrared rays with a first wavelength to emit infrared rays with a second wavelength, and
the first wavelength is longer than the second wavelength.

10. The test strip of claim 1, wherein:
each of the conjugation pad, the membrane, and the absorption pad comprises a solid-phase capillary support, porosity of the sampling pad is greater than porosity of the solid-phase capillary support.

11. The test strip of claim 1, wherein:
a surface of the sampling pad is treated with a mixed solution of PVP, sucrose, BSA, and Tween 20.

12. A microbial sensor device comprising:
an air sampling device configured to collect air;
a combined sampling/diagnosis kit positioned on an upper surface of the air sampling device to collect a specimen from the air sucked by the air sampling device; and
an analysis device configured to irradiate the combined sampling/diagnosis kit with infrared rays with a first wavelength to receive the infrared rays with a first wavelength from the combined sampling/diagnosis kit, thereby detecting a target material from the specimen,
wherein the combined sampling/diagnosis kit comprises:
a porous sampling pad configured to collect the specimen from the air passing through the corresponding one air suction port;
a conjugation pad which is positioned in contact with the sampling pad, and in which a plurality of capture agent-nanoparticle composites specifically binding to the target material are dispensed;
a membrane which is positioned in contact with the conjugation pad, and comprises a test line to which the capture agent-nanoparticle composites to which the target material binds are conjugated when the specimen moves, and a control line to which the capture agent-nanoparticle composites are conjugated; and
an absorption pad configured to absorb the moving specimen.

13. The microbial sensor device of claim 12, wherein:
the air sampling device comprises:
an air suction port device configured to provide an air path through which air flowing in through an air suction port of the combined sampling/diagnosis kit flows;
a hollow air suction device configured to collect the air passing through the air path; and
an air suction fan device configured to produce an air suction force,
wherein the sampling pad is positioned in the air path.

14. The microbial sensor device of claim 13, wherein:
the air suction port device comprises:
an air outlet formed in a position corresponding to the air suction port of the combined sampling/diagnosis kit; and
an air suction through hole formed to extend from the air outlet,
wherein the sampling pad is positioned on the air suction through hole.

15. The microbial sensor device of claim 13, wherein:
the hollow air suction device comprises:
a first circular aperture formed on an upper surface thereof;
a second circular aperture formed on a lower surface thereof; and
a common suction space formed between the first circular aperture and the second circular aperture.

16. The microbial sensor device of claim 15, wherein:
the common suction space comprises:
a circular cylinder space having a predetermined depth from the first circular aperture; and
a space having a trapezoid circular cylinder shape between a third circular aperture positioned in a lower portion of the circular cylinder space and the second circular aperture.

17. The microbial sensor device of claim 13, wherein:
the air suction fan device comprises:
a fan configured to produce the air suction force while rotating; and
an outlet configured to discharge the air sucked by rotation of the fan.

18. The microbial sensor device of claim 12, wherein:
the kit further comprises:
a cartridge configured to receive a test strip in order to couple the test strip, which comprises the supporter, the sampling pad, the conjugation pad, the membrane, and the absorption pad, to the air sampling device.

19. A method of detecting a microorganism using the kit coupled to the air sampling device, the method comprising:
collecting a specimen from air sucked by the air sampling device using a porous sampling pad through which the air passes;
dispensing an analysis solution in the sampling pad to move the specimen thereto;
conjugating a target material included in the specimen to one of a plurality of capture agent-nanoparticle composites in a conjugation pad;
conjugating a capture antibody fixed in a membrane to the target material of the capture agent-nanoparticle composites to which the target material binds as the specimen moves; and
absorbing the moving specimen using an absorption pad.

20. The method of detecting a microorganism of claim 19, further comprising:
conjugating at least one of the plurality of capture agent-nanoparticle composites to a control antibody fixed in the membrane.
